(19) United States
(12) Patent Application Publication
(10) Pub. No.: US 2026/0315564 A1
(43) Pub. Date: Oct. 15, 2026

(54) THE USE OF TP3 (THYMOPENTIN ANALOG) BY INCREASING THE ACTIVITY AND NUMBER OF T-CELLS INVOLVED IN THE DEFENSE MECHANISM OF THE IMMUNE SYSTEM OF MAMMALS, FOR THE PREVENTION OR TREATMENT OF DISEASES TO EXTEND THE AVERAGE LIFESPAN

(71) Applicant: László DÉNES, Budapest (HU)

(72) Inventor: László DÉNES, Budapest (HU)

(21) Appl. No.: 19/589,290

(22) PCT Filed: Oct. 19, 2024
(86) PCT No.: PCT/HU2024/050081

§ 371 Date: May 21, 2026

(30) Foreign Application Priority Data
Oct. 19, 2023 (HU) P2300355

(51) Int. Cl. A61K 38/06; A61P 37/04

(52) U.S. Cl. CPC A61K 38/06; A61P 37/04

(57) ABSTRACT

The goal of the therapy is to restore the balance of the immune system, rehabilitate patients with reduced immunity, and prolong the average age by maintaining immune activity by using Thymotridan (TP3/tripeptide), the smallest active part of the thymus hormone Thymopoietin (49 amino acids). Synthetic TP3 appears with two peaks in a wide dose range. There are differences of several orders of magnitude between the two effects, which, separately or alone, provide the “enhancer” effect endogenously and result in the renewal of the immune system. Thymocartin (TP3) can be used for prevention or causal therapy by increasing the activity and number of lymphoid and myeloid cells. Thymocartin increases the activity of the immune system of healthy individuals or the elderly, and the lifespan extend. The role of TP3 is primarily to improve the balance of the system by promoting the maturation and increasing the activity of immune cells in patients with immunodeficiency (autoimmune, cancer) and by replacing helper and suppressor cells. Infection can be eliminated in juveniles due to their weaker immune activity by TP3 therapy.

### THE SUBJECT OF THE INVENTION

The invention aims: to prolong the average age by TP3 (thymopentin analog) treatment. The use of TP3 (Thymotridan), an analog of Thymopentin, which endogenously triggers the renewal (rejuvenation) of immune cells and affects the activity of the precursor and mature lymphoid and myeloid cells, can be combined with suitable medical substances.

### FIELD OF APPLICATION

The invention also applies to drugs for the treatment and prevention of various immune diseases (AIDS, various tumour diseases, autoimmune processes), but also for use in particular to extend the average age, as well as procedures for these.

### Clinical Studies of Thymopentin Include:

hepatocellular carcinoma
thymopentin for patients suffering from HIV-1 infection,
treatment/prevention of liver tumour/HIV infections were aimed at its application.

### STATE OF THE ART

Many thymus hormones are known, and some have been put into clinical use in synthetic form. These include the thymus hormone, called Thymopoietin discovered by G. Goldstein et al. (Nature 1247, 11, 1974), and the synthesized active group thymopentin (TP5) consisting of five amino acids. Thymopentin primarily induces the differentiation of T cells (Schlesinger et al.: Cell 5,631,1978). During the tests, TP5, further shortened at the N-terminal end of the molecule, lost its activity (Abiko et al. Chem Pharm Bull 28, 2507, 1980.) Thus, the research group developed it into a drug and conducted clinical trials on autoimmune patients/RA and tumour patients/50 mg injection in the form of/performed with TP5.

At the Köbánya Pharmaceutical Factory (Richter Gedeon Rt, Budapest), the TP5 analogs were obtained by shortening the C-terminal end of the hormone: no. 168,431. oligopeptides consisting of 4 amino acids (TP4) and 3 amino acids (TP3) as described in a Hungarian patent (Kisfaludy et al.; Tetrahedron Let. 1785,1974). These molecules, like TP5, 50 mg in (iv., sc.) were included in clinical Phase I and Phase II. for examination. The final preclinical and clinical WO 2025/083437 2 PCT/HU2024/050081 (phase II.) studies were carried out with the highly pure, crystallized products (prepared and registered by the Hungarian Institute of Inventions No: 210 721 A9).

Chinese Patent Application No. CN115536732 (A) relates to thymopentin conjugated with beta-glucan as an immunomodulatory and anti-tumour drug.

Chinese Patent Application No. CN111494600 (A) covers pharmaceutical and medical preparations in immunodeficiency diseases.

The drug composition contains thymopentin and thymalfazin, it can significantly enhance cellular and humoral immunity of deficient immunosuppressive/organisms/,/low dose and low frequencies/, and improve the appropriateness of the patient's clinical value. Thymopentin thymalfazine enhances the body's immune function.

The state of the art also includes the clinical studies carried out and published below, where the treatment of tumour patients with thymus peptides was compared in a meta-analysis study (Wolf et al., Cochrane Database of Systematic Reviews 2011-2017, Issue 2. Art. No: CD003993). Randomized clinical trials were used with synthetic peptides, in 26 studies involving 2,736 patients with metastatic melanoma showed promising results in reducing severe infections when radiation or chemotherapy was used in parallel with TP5 treatments. On the other hand, the number of survivors was not significantly affected by the treatments. We have recognized that certain medicinal products containing TP3 (thymopentin analog) increase the renewal and activity of the body's defence system.

Tests presenting the novelty related to the invention:

TP3 produced a surprising and non-expected result during the dose-effect relationship tests. The compound instead of the usual single-peak effect, which is also mentioned as a well-known fact in the pharmacology handbook: (L. B. Buxton: Sec.1. General Principles of Pharmacokinetics and Pharmacodynamics, in Goodman& Gilman's: Pharmacol Basis of Therapeutics, 12th. Ed Mc Grandhill, Med Publ Div, New York, pp. 33-34, 2006), produced two peaks over a wide dose range. One effect was detected in a physiological dose range, the other appeared at several orders of magnitude lower doses (1000 times less). Effects of this type, showing a dose-response curve with two peaks (bimodal), were also observed by other researchers on other cells, with Deprenyl/Selegiline in the central nervous system (Knoll et al., Arch Int Paharmacodyn Ther, 328,1-15, 1994; and similar with trace amines (Leo et al.: Neuro Pharmacol, 81,283-291, 2014.). Inventors also obtained such a bimodal dose-response curve with Deprenyl/Selegiline on human brain vascular endothelial cells (Dénes et al.: Life Sci. 79, 1034-1039. 2006).

The effect of Thymotridan appeared with a bimodal pattern in the immune system similar to Deprenyl in the central nervous compartment.

The body has endogenous hormone-like “enhancers” effect such as the thymus hormone in the immune system and in the central nervous system (B-Phenylamine/PEA, dopamine, traceamines), which are responsible for the maturation and rejuvenation of various neuronal cells. Knoll et al. called this type of effect on neural cells the “enhancer” effect, which ensures the continuous activity of the neuronal system. The inventors are the first to describe the enhancer effect on the immune system.

Likewise, it is the first report that thymopentin analogs have a regenerative effect on the monocyte-macrophage (myeloid) system.

### The Essence of Recognition

The bimodal dose-effect curve can be separated, with a different character, and a distinguishable double effect. based on two receptors. The phenomenon can be attributed to the non-specific effect on the physiological amount, which causes an increase in the activity of mature cells. With very low doses, it is specific, trying to renew the cells (lymphoid and monocyte/macrophage), maturing and stimulating the cells. They generate “enhancer” effects. Cell maturation can be due to the “noise level” either parallel to the cell activation or independently of it. The two effects of different nature and quality exert their effects by binding to two completely different surface structures, or distinct phenotypes at a different stage of the maturation process. Both effects, separately or together, are involved and play an essential role in the restoration of the balance of the immune system.

Other effects: The research results of previous years proved that the body's defence mechanism depends on the thymic hormones. They activate the reactions of lymphocytes and increase the number of cells participating in the maturation process. We have to calculate the activation of lymphoid and myeloid cells, which will be discussed in detail later on. Thymic hormones are secreted into the circulation and integrated into the endocrine corticosteroid system with their exocrine effects. Thymus hormones have multidirectional effects, they play an important role in inflammatory processes by producing proinflammatory and inflammatory factors. In addition to the thymus hormone-like effects of the oligopeptide TP3, they focused on testing the effects in a wide range of doses. Thymopentin (TP5) is included in the tests as a control standard compound.

Thymopentin (TP5) and its fragment TP3 (Richter) (Kisfaludy et al. 1983) were used in the amounts described in the individual methods for our experiments establishing the protective circle.

These proteins (TP5 and its fragments) in very high doses (ED50>2000 mg/kg), even during 12 months of use, did not cause any signs of side effects (in animals). Similar results were obtained with TP5 and applied to patients for 12-24 months (it proved to be a well-used, safe drug candidate (Friedmann: Surv Immunol Res, 4, suppl. 1, pp. 139-148, 1985).

### Research Materials and Methods

Blood from donors was used for our experiments, where the blood samples were used either from animals or human donors. The heparinized blood samples were separated on a (FicolUromiro/Pharmacia-Bracco) gradient (Boyum 1968). 2×106 and 107 lymphocytes/ml cells were tested in E-rosette tests, the blood donors were the following animals:
Rat: Hannover Wistar (Richter) newborn rats were used to study the maturation of the immune response. As our mothers, 12 (maximum) randomized newborns were placed (21±10C, humidity: min. 65%) in a test litter.
   Guinea pig: Donkin-Hartley (Lati) 250-300 g male animals were included for the DTH reaction test.
   2.3.3/Mice: Mouse strains of different genetic backgrounds were used: CFLP (outbred) from the Lati stock), Balb/c (inbred) and Balb/c nu/nu mice from the SzBC/Szeged stock.

### EXAMPLES

### In Vitro Studies

A./E-rosette test
B./Azathioprine inhibited rosette test:

In a preliminary study, the ED75 inhibitory effect of azathioprine was determined (Verhagen et al., 1980) and 500 μg/ml was used during the studies. An equal volume (200 μl) of activityinhibiting and isolated cell suspension and peptides (Oct. 3, 2010-15 mol/1) were measured. After 1 h (370C) incubation, the cell suspensions received 200 μl of 1% SRBC suspension and then centrifuged (90×g 5 minutes), 400 μl of the supernatant was aspirated.

The number of rosette-forming cells was counted. The activity was determined as percent recovery (R %). then it was centrifuged (90×g for 5 minutes), and 400 μl of the supernatant was aspirated.

$$X \% = 100 / ExpERFC - AzERFC$$

### ERFC-AzERFC

### C./Active Rosette Test:

The test was carried out according to the method of (Wybran and Fundenberg 1973). 107/ml lymphocytes from either healthy blood donors or autoimmune patients were included in the tests. The effect of the substances was tested in the presence of 100 μl of Oct. 3, 2010-15 mol/L. test compound. After an incubation period, 100 μul of cell suspension was placed on a glass slide, fixed for 3 minutes (with 50 μl of 3% glutaraldehyde solution), and stored in a humid chamber until evaluation.

### D./Cell Wall Marker Test

D.1/Direct cytotoxicity test/for the detection of Thy, Lyt antigens (Fathman 1979, in Immunol Meth I. Eds. Lefkovits, Pernis, Acad Press, New York, pp. 212-213.) was used. Subpopulation ratios were determined from bone marrow cells, thymus, and spleen cells isolated from the lymphatic organs of mice. The cell suspensions (1.087-1.088 g/cm3) were centrifuged ona density gradient (2000 rpm, 20 minutes), and the cells were washed 3× with RPMI1640 medium containing 0.3% BSA, then adjusted to 107/ml. Thyl, Lyt 1, Lyt2 monoclonal antibodies (McAb/Ando I. (ICRF Human Tumor Immunol. UCM Med Sch. London) were obtained. 25 μl McAb and 25 ul cell suspension were incubated (60′, 40C1), after centrifugation (1250 rpm 10′), the cells were resuspended with 40 μul of diluted (1:50) rabbit complement, then incubated (370C, 60′). The cell suspensions were stained with 1:5 dilution of Eosin Y (Jansen). the ratio of live to non-living cells was determined.

### In Vivo Studies

In this experiment according to Example 3, the maturation of the immune response was investigated in newborn rats (Ceglovszki, LaBadie, 1975).

Rats (Hannover Wistar) were treated (i.p.) within 6-12 hours after birth. Treatments after birth in a single session or in another examination
on the 7th day after the first treatment;
another group was vaccinated 3 times a week after the first day of 50. treatment
in a subsequent group treated over three weeks (9× in total) (10-3,10-5,10-7//10-9, 10-11,10-12, 10-13 M/animal, i.p).
in the fourth test series, the animals were treated with TP fragments at 0, 1, 2, 4, or 5 days of age. The rats were unanimously immunized on the 14th day after birth (1% BVVT with 0.5 ml cell suspension, i.p.). Immune responses were measured after 7 days on day 21 of the experiment.

### In Vivo Studies

In this experiment according to Example 3, the maturation of the immune response was investigated in newborn rats (Ceglovszki, LaBadie, 1975).

Rats (Hannover Wistar) were treated (i.p.) within 6-12 hours after birth. Treatments after birth in a single session or in another examination
on the 7th day after the first treatment;
another group was vaccinated 3 times a week after the first day of treatment.
in a subsequent group, the treatments took place over three weeks (9× in total) (10-3,10-5,10-7//10-9, 10-11,10-12,10-13 M/animal, i.p.).
in the fourth test series, the animals were treated with TP fragments at 0, 1, 2, 4, or 5 days of age. The rats were randomly immunized on the 14th day after birth (1% BVVT with 0.5 ml cell suspension, i.p.). Immune responses were measured after 7 days on day 21 of the experiment. 1.2. Determination of anti-BVVT Ab titers (from rat blood samples ex vivo)
with the haemagglutination method (Takátsy and Fürész, 1955) on 96-well plates (Flow). 50 ul of 0.9% NaCl solution was added to each well. The sera (50 μl) were diluted by half dilution in 12 steps, mixed with 25 pμl 2% BVVT cell suspension, and incubated (1 hour at 370C). The hemagglutination titers of the sera are determined. Values expressed in logarithmic values refer to total agglutination.

1.3./Determination of the number of plaque-forming cells (PFC) (using rat spleen cells) The chopped splenocytes were placed in a culture medium (Parker 199) and then shaken with a shaker for 1 minute. The cells were suctioned from the surface of the rapidly settling tissue debris. Red blood cells were removed osmotically. Cell suspensions were adjusted to 107/ml. The cell suspensions were mixed with a 15% BVVT cell suspension in a ratio of 2:1. PFC was determined with the mixture using a modified chamber technique according to Channingham et al., 1966). Before filling the chamber, 50 μl of fresh diluted guinea pig serum (1:20) was added. The chambers (2-2/slide) were incubated (370C 45′) after sealing with paraffin, the formed plaques were counted under a microscope (at 400× magnification) within 2 hours Marker tests related to the rejection reaction of an adult thymus-depleted mouse skin graft 1.4./Rejection of skin transplants in nude mice after treatment with TP fragments (Snell et al., Transplantation 5, 492, 1967).

After thymectomy, mice were treated with doses of 1 mg/kg, i.p. once a week for six weeks. Thy 1+, Lyt1+, and Lyt2+ in the spleen cells of mice at the age of 13 weeks cell proportions were determined based on what was previously described.

Immune reactions of Balb/c nu/nu mice in response to TP-fragment treatment.

Mice were treated for 4 weeks 3 times a week (every other day) with TP fragments (10-9, 10-8, 10-7//10-3, 10-2, 10-1 M/animal ip. (Veys et al., 1983) at the end of the treatments (week 3), the mice were immunized (with 0.5 ml 1% BVVT suspension ip. The immune response was expressed in antibody titers as above).

A cell suspension of 107 cells/ml was prepared from the spleen cells of Balb/c nu/nu mice treated with TP fragments in RPMI 1640 (with 10% horse serum). ConA (Pharmacia) and PHA (Bacto) reagents were used as inductive agents. In the case of ConA, the exposure lasted 24 h, while the PHA exposure lasted 72 h. 4 hours before the end of the culture, indicated Thymidine was added to the culture (1 μCi 3H-Thymidine/in 10 μl/well). −48 cells were collected on glass fiber discs (Whatman G and incorporated F/C). After washing and drying the disks (600C, 30′), they were placed in scintillation liquid and the incorporated 3H-Thymidine hit number (cpm) was recorded.

### 2./Effect on the Maturation of the Immune Response

2.1./In delayed-type hypersensitivity reaction (DTH) and Macrophage disappearance reaction (MER) in guinea pigs
2.1.1./In the DTH tests, male guinea pigs (300-350 g Donkin-Hartley/Lati) were immunized 4 weeks before the test (4 soles 100 μg/sole BCG/OKI) mixed with complete Freud Adjuvant (Difco 1:4). The animals were treated with TP fragments (1 mg/kg/ip.) 3×/week for two weeks. The extent of the local DTH reaction was characterized by the magnitude of the redness obtained after administration of 10 IU of tuberculin (for 18-48 hours). The local reactions were induced by the combined administration of the oligopeptide to be tested (10-5, 10-7, 10-9 or 10-4, 10-6, 10-8 M/treatment) and PPD.
2.1.2./According to the macrophage disappearance reaction (MER) tests (Nelson and Boyden, 1963). Guinea pigs were immunized as described above. To increase the number of peritoneal macrophages, 10 ml snail glycogen (1 μg/ml, i.p./Sigma) was given on day 24 of immunization. On the 28th day of the experiment, the animals received a second antigen stimulus (PPD/Human Int.). After 4 hours, the animals were bled and the abdominal cavity was washed twice with a 0.9% NaCl solution containing 50 ml of heparin (10 NE/ml/
G. Richter). They were centrifuged at 500 g for 5 minutes, the cells were suspended in 2 ml of physiological NaCl solution. The total cell count in the peritoneum was determined. After Gimsa's painting of the number of macrophages in 100 PEC cells was characterized by nonspecific esterase staining.
3./Monocyte-macrophage identification by non-specific esterase staining
12 mg Naphthol AS acetate (Sigma) was dissolved in 20 mg/l ml acetone (Reanal), then 20 ml 0.1 M phosphate buffer (pH=7.2) was added and homogenized with 1 ml/20 mg Echtrot K1 saline solution; the supernatant was filtered from the pink precipitate. 0.5-1.0 ml of centrifuged liquid was added to a sediment of 2×105 cells and incubated (30′ RT). After washing with 0.9% NaCl solution, the ratio of pink (macrophages) and green cells is determined.

### Results

### Example 1

Effects of inducing renewal of the immune system and increasing activity in Balb/c nude mice The nude mouse is genetically deficient in the thymus. The mammary gland atrophies over time after hormonal follow-up in mice and humans. As a result, the strength of the lymphoid, and within that of the T cells, decreases. All this means a definitive decrease in antigen recognition capacity. The increasing number and activity of immune cells in the thymus of nude mice is increasing health and quality of life span.

### 1./Table

The number of Thy1+ cells in the spleens of Balb/c nu/nu mice after TP-fragment treatment (Thy 1+ marker-bearing cells in mice correspond to human lymphocytes)

| M/mice | 10⁻⁹ | 10⁻⁸ | 10⁻⁷ | // | 10⁻³ | 10⁻² | 10⁻¹ |
|---|---|---|---|---|---|---|---|
| TP5 | 9.5 | 79* | 125** | | 94* | 129** | 131* |
| TP3 | 10 | 61* | 69* | | 85* | 81* | 71* |

n = 8/group
Control Thy 1+ = 5.4
* = p < 0.05,
** = p < 0.01 Student's t-test

### 1.2./Antibody Production in Balb/c Nu/Nu Mice Treated by TP3

| M/eger | 10⁻⁹ | 10⁻⁸ | 10⁻⁷ | // | 10⁻⁴ | 10⁻³ | 10⁻² |
|---|---|---|---|---|---|---|---|
| TP5 | 14.5 | 19.9 * | 24.6 ** | | 11.9 | 19.8 | 26.4 ** |
| TP3 | 4.0 | 5.3 | 3.3 | | 4.0 | 5.1 | 4.8 |

n = 10/group;
Control < 3.0
* = p < 0.05,
** = p < 0.01 Student's-t test

1.3./the Number of Plaque-Forming Cells (PFC) in (Balb/c Nu/Nu Mice) after TP-3 Treatment

| M/eger | Kontroll | 10−8 | 10−7 | 10−6 | // | 10−3 | 10−2 | 10−1 |
|---|---|---|---|---|---|---|---|---|
| TP5 | 55.7 | 9.5 | 71.0* | 72.5* | | 67.4 | 73.0* | 76.5* |
| TP3 | 53.0 | 10 | 66.4* | 79.8* | | 86.5** | 76.1* | 69.2* |

n = 8/group
* = p < 0.05,
** = p < 0.01 Student's t test

To achieve an increase in the number of PFC cells, an order higher dose is required than to activate already mature cells. As a result of the treatment, the number of PFC cells increases by

(TP3>TP5=47-35%/10-3. M/mouse/i.p.). TP3 enhances the strengthening of immune cells, while TP5 increases the activity of current antibody-producing cells. 1.4./Changes in Lyt 1+ (helper) and Lyt 2+ (suppressor) cell ratios in Baalb/c nu/nu mice To better understand the TP effects, we also examined changes in the proportion of cell populations.

Effects on Helper/Suppressor Cell Ratio (in Balb/c nu/nu mice)

Thy1+ (%) Lyt 1/Lyt 2

| Vegyületek | Csontvelő | Lép | Csontvelő | Lép |
|---|---|---|---|---|
| Kontroll | 3.7 | 5.4 | 0.97 | 1.10 |
| TP5 | 47.3 | 71.0 | 1.95 | 2.48 |
| TP3 | 56.2 | 117.1 | 0.64 | 2.89 |

n=8/group; Treatment: 10-5 M/mice/ip every other day for 3 weeks (9×/ip.)

TP-fragment treatments also initiate changes in the bone marrow and spleen (Table 1.3). T3 induced an activating effect in both compartments. The effects were stronger in the Spleen. In the spleen, the treatments (TP3 and TP5=170>130%) produced a greater change than in the bone marrow. The effect of TP3 was also significant in the bone marrow. The helper/suppressor (Lyt 1+/Lyt2+) cell ratio increased significantly in both the spleen and the bone marrow.

In a genetically deficient thymus system, TP3 treatment compensates for the antibody production and the number of cells responsible for the immune activity resulting from the functional deficiency of the immune system. The enhancer-like effect of the preparation ensures
the continuous renewal of the immune system (rejuvenation).

### Example 2

Effect of TP3 on immune activity and survival of aged mice

### 2./Table

TP effect on antibody production and PFC cell count in aged mice (Balb/c)

PFC IgM/logx²

| | | |
|---|---|---|
| Kontroll^{§} | 6.3 | 84.1 |
| Idős | 3.7 | 41.0 |
| I + TP5 | 6.1* | 44.7 |
| I + TP3 | 4.0 | 59.9* |

^{§}Mice/ Control: 3 Months old; Aged: mice: 18 Months old
n = 8/group;
* = p < 0.05 Student's t-test

### 2.2./Examination of the Effect on the Lifespan of Old Mice

Lifespan study of Balb/c male mice with TP3 treatment (3×/week)

The treatment started before the mice were 18 months old (72 weeks) and the control lasted until the destruction of the group (until the 86th week/21st month, over 3 months).

| Weeks | Control | 1.0 mg/kg/íp. | 0001 mg/kg/ip. | Kezelés |
|---|---|---|---|---|
| 79 | 10 | 10 | 10 | + |
| 80 | 8 | 10 | 10 | + |
| 81 | 8 | 10 | 10 | + |
| 82 | 7 | 10 | 10 | + |
| 83 | 5 | 10 | 10 | + |
| 84 | 3 | 10 | 10 | + |
| 85 | 3 | 10 | 10 | + |
| 86 | 0 | 10 | 10 | + |
| // | // | // | // | |
| 104 | | 10 | 9 | |
| 105 | | 7 | 6 | |
| 106 | | 6 | 3 | |
| 107 | | 2 | 2 | |
| 108 | | 1 | 0 | |
| 109 | | 0 | | |

The lifespan of mice has been compared to the general lifespan of humans. They found that mice mature 150 times faster than humans from birth to 1 month old. Mice grow 45× faster in 1-6 months, while this rate of development in humans shows only a 25-fold difference in old age. (J. G. Fox (ed) American College of Laboratory Animal Medicine series, Elsevier, AOP Amsterdam, Boston, 2007.) As a result, old age is reached in mice at 12-18 months, while in humans this disease occurs at 56-64 years.

In our studies, the treatments with TP were started when the animals were 1.5 years old and continued until the mice of the control group died, i.e. until the 12th week, for 3 months. There

was no difference in the survival rate of the treated groups. The animals outlived the control mice by more than 1.5 months (in the absence of further treatments). The 8-week treatment in mice can be considered almost five years of human survival.

In summary, the studies indicate that the average life can be extended with the preventive therapy of TP3.

### Example 3

The effect of TP3 on the “enhancer” processes that induce immune response and support regeneration in neonatal rat

Enhancement of primary antibody production in neonatal Hanover (H) Wistar rats Antibody production in neonatal rats is low in the first few weeks after birth. In this case, a significant antibody-enhancing effect was obtained in rats treated with doses of the TP-3 (1×/ip.

rat) on a single occasion. The number of antibody-producing cells increased significantly compared to control/untreated animals (Table 3.1.)

After the 9× treatment, the bimodal effect became even more pronounced.

### 3./Table

Anti-BVVT-Ab titers (IgM) 2 log; Newborn after TP3 treatment (1×/ip.) in H. Wistar rats

| TP | Contr. | 10⁻¹³ | 10⁻¹¹ | 10⁻⁹ | // | 10⁻⁶ | 10⁻⁵ | 10⁻⁴ | 10⁻³ |
|---|---|---|---|---|---|---|---|---|---|
| TP5 | 6.3 | 7.0 | 7.7 | 8.0* | | 8.5* | 8.6* | 8.1* | 7.6 |
| TP3 | 5.2 | 7.0 | 7.5 | 8.5* | | 7.6 | 7.4 | 7.2 | 7.0 |

n = 12/Group;
* = p < 0.05 Student's t-test

3.2. Anti-SRBC-Ab Titer (IgM) 2 log; after TP3 9× (3×3 Weeks/Ip.)

| TP | Contr. | 10⁻¹³ | 10⁻¹¹ | 10⁻⁹ | // | 10⁻⁶ | 10⁻⁵ | 10⁻⁴ | 10⁻³ |
|---|---|---|---|---|---|---|---|---|---|
| TP5 | 6.3 | 7.9* | 10.7* | 7.2 | | 7.5 | 8.6* | 8.2* | 7.3 |
| TP3 | 5.2 | 6.9 | 8.2* | 6.8 | | 6.6 | 7.3 | 7.7* | 7.0 |

n = 12/Group
= p < 0.05 Student's t-test

### 3.3. a the Effect of TP Treatment on PFC (Plaque-Forming Cells) in H. Wistar Rats (1×/Ip)

| TP | 10⁻¹³ | 10⁻¹¹ | 10⁻¹⁰ | 10⁻⁹ | 10⁻⁸ | 10⁻⁷ | 10⁻⁶ |
|---|---|---|---|---|---|---|---|
| TP5 | 67 | 75 | 76 | 90* | 148* | 129* | 71 |
| TP3 | 59 | 75 | 102* | 73 | 150** | 128* | 90* |

Control = 53.9 ± 8.8;
n = 12
* = p < 0.05,
** = p < 0.01 Student's t-test

### 3.3.B./Effect of TP-Treatment/9× (3×3 Weeks/Ip)/on the Number of PFC

| TP | 10⁻¹³ | 10⁻¹¹ | 10⁻¹⁰ | 10⁻⁹ | 10⁻⁸ | 10⁻⁷ | 10⁻⁶ |
|---|---|---|---|---|---|---|---|
| TP5 | 143 | 162 | 185* | 126 | 223* | 203* | 159 |
| TP3 | 147 | 158 | 176* | 159 | 200* | 191* | 159 |

Controll = 139 ± 12.7;
n = 12/ group;
* = p < 0.05 Student's t test

TP fragment treatments significantly increased the number of PFC cells. The degree of growth became even more pronounced after 9× treatment. The dose-effect relationship after both treatment regimens shows bimodal forms. TP3=TP5 potency could be observed. The studies make it clear that the enhancer-like effect of TP-3 can strengthen the immune system's activity. An Example is young patients against diseases (infections).

### Example 4

Investigation of clinical-oriented effects/ex vivo on healthy human cells

Dose (mol/l)

| TP | Contr. | 10⁻¹⁴ | 10⁻¹³ | 10⁻¹² | 10⁻¹¹ | // | 10⁻⁹ | 10⁻⁸ | 10⁻⁷ | 10⁻⁶ | 10⁻⁵ |
|---|---|---|---|---|---|---|---|---|---|---|---|
| TP5 | 27.8 | 26.1 | 25.9 | 28.4 | 25.5 | | 26.1 | 27.1 | 29.4 | 24.4 | 26.9 |
| TP3 | 30.3 | 27.1 | 39.8* | 28.7 | 25.5 | | 35.1 | 41.7** | 38.6* | 39.3* | 37.9 |

### 4.2./Active E-Rosette Forming Cells in the Presence of the TP Analog (Mol/L)

Dose (mol/l)

| TP | Contr. | 10⁻¹³ | 10⁻¹¹ | 10⁻⁹ | 10⁻⁷ | 10⁻⁵ |
|---|---|---|---|---|---|---|
| TP5 | 6.3 | 7.9* | 10.7* | 7.2 | 7.5 | 8.6* |
| TP3 | 5.2 | 6.9 | 8.2* | 6.8 | 6.6 | 7.3 |

Control = 139 ± 12.7;
n = 12/ group;
* = p < 0.05 Student's t-test

Lymphocytes from healthy individuals were ineffective against TP5 treatment, while TP3 was
active in both physiological and low dose ranges.

Active E-rosette formation: both TP fragments showed activity in the lower dose range. Based
on all of this, the activity of healthy lymphocytes can be activated at a lower dose, which makes the TP3 fragment suitable for strengthening the immunity of patients facing surgery with preventive therapy

### Example 5

Examination of complex immunological effects in a system with reduced immune activity, improvement of the helper/suppressor ratio

### 5. The Effect of TP3 was Tested on the Activity of the Immune System by Rejection of Skin Graft

in adult thymectomized (Tx) C57BL mice.

Lymphocytes from healthy individuals were ineffective against TP5 treatment, while TP3 was
active in both physiological and low dose ranges.

In the active E-rosette test, both TP fragments showed activity in the lower dose range. Based on all of this, the activity of healthy lymphocytes can be activated at a lower dose, which makes
the TP3 fragment suitable for strengthening the defenses of patients facing surgery with preventive therapy.

### Table 5.

Adult thymus-removed (Tx) mouse spleen cell subpopulations after TP treatment (1.0 mg/kg/ip.) (%)

cells in %

| | Thy 1 | Lyt 1 | Lyt 2 | Lyt1/Lyt2 |
|---|---|---|---|---|
| Kezelés | male | male | male | male |
| Control | 30.1 | 24.3 | 13.7 | 1.77 |
| Tx | 8.1 | 18.8 | 9.6 | 1.96 |
| Tx + TP5 | 21.2 | 23.2* | 14.2* | 1.63 |
| Tx + TP3 | 25.3 | 25.7* | 13.8* | 1.86 |

TX = thymus-removed n = 8/group;
*p < 0.05 Student's t-test

In thymus-less mice (Tx mice) the number of T cells significantly decreased (below 5-10%). As a result of TP3, the number of cells normalized (to 25-30%) TP3 significantly increased the
number of helper and suppressor cells.

### 5.2./Rejection of the Transplanted Skin Graft in an Adult Thymus-Removed (Tx) Mouse

In adult thymus-ablated mice, the transplanted skin graft is rejected later than in mice with thymus.

| Treatment | male | Female |
|---|---|---|
| Contr./Untreated | 27.8* | 14.5** |
| Tx C57BL | 59.8 | 54.6 |
| Tx + TP5 | 41.3 | 23.1** |
| Tx + TP3 | 28.4* | 34.4* |

n = 8/group;
§ = compared to Tx control;
*p < 0.05;
**= p < 0.01 Student's t-test

In the case of female mice, TP3>TP5, while in the male group, TP5>TP3 treatment enhanced the recovery of T cells and thereby shortened the skin graft survival time.

### Example 6

Monocyte-macrophage maturation and activation is the renewal of reduced immunity DTH reaction (delayed-type hypersensitivity reaction)

### Table 6.

Study of the effect on delayed-type hypersensitivity reaction (DTH) in guinea pigs.

(Antigen = Ag; Agl = (BCG sc); Agll = (50NE PPD ic);
DTH-reakció, bőrpir mm/π
0.9% NaCl

| | |
|---|---|
| PPD | 217.9 |

| Treatm. mg/kg/s.c. | 0.1 | 1.0 | 20.0 |
|---|---|---|---|
| TP5 | 127.7* | 158.1 | 194.6 |
| TP3 | 138.6* | 89.3** | 186.0 |

n = 12/group;
*p < 0.05 = X² próba,
**p < 0.01 = X² próba

Peritonealis Macrophages in DTH-reaction
tengeri malacokin guinea pigs

| Treatm./Staining | Nonspecific esterase | Giemsa |
|---|---|---|
| 0.9% NaCl | 36.7 | 58.7 |
| PPD | 14.0 | 19.0 |
| TP3 | 28.3* | 55.0** |

Similar results were obtained if the number of macrophages that appeared in the peritoneum after the treatments were counted after staining:

With both staining, the largest increase in the number of macrophage cells was obtained with the 1.0 mg/kg drug dose.

### 6.2./Examination of the Effect on the Phagocytic Function of Peritoneal Macrophages CFLP Outbred in Mice

Macrophage phagocytosis ex vivo (X=yeast/100 cells)

TP-treatment

$$80 mg / kg / CY + after 6 \text{h/} TP / mg / kg / sc$$

$$\begin{matrix}Makrohage phagocitosis ex vivo & = & particl . / 100 cells ) \\ TP & & TP - treatm .\end{matrix}$$

$$80 mg / kg / CY + 6 h / TP / mg / kg / sc$$

| | Untreated | CY | | | |
|---|---|---|---|---|---|
| TP-k | Contr. | p.o. | 0.1 | 1.0 | 10.0 |
| TP5 | 194.5 | 111.5* | 149.6 | 123.1 | 95.6 |
| TP3 | 194.5 | 111.5* | 227.8 | 246.3* | 1776.0 |

n = 15/csoport;
*p < 0.05 Student's t test

In Outbread mice, CY treatment significantly inhibited the phagocytic activity of macrophages.

TP 3 1.0 mg/kg/sc treatment prevented it, while TP5 was ineffective against the immunosuppressive effect of CY.

### Example 7

Renewal of the immune system with a significantly reduced helper/suppressor cell ratio with TP3 treatment, increasing the number of suppressor cells (in Autoimmune, Tumor patients) 7.1./Comparative study of the effect of TP treatment on white blood cells with differently I nhibited activity (Li, MN, PMN/Maximum recovery in control %)

RA HEAD-NECK CANCER

| | | | | | | | |
|---|---|---|---|---|---|---|---|
| TP-k | Contr. | | +AZ | SP | SP + PPD | SP | SP + PPD |
| | Li | Li | Li | MN | MN | Li | Li |

Normál//Rheumatoid Arthritis Fej-nyak tumoros beteg
Limfocita Limfocita Limfocita MN MN Limfocita
p<0.05; ** p<0.01 by Student's t test compared to the control
Human cells: RA=Rheumatoid Arthritis; Tumor: head and neck cancer
Az Azathioprine/75 μg/ml
Cells: Li=Lymphocyte; MN=mononuclear; PMN=Polymorphonuclear

$$SP = Cell + TP treatement ; + PPD = + PPD / Antigen + TP treatement$$

TP5 was ineffective on lymphocytes from healthy individuals, while TP3 was active. The activity of lymphocytes inhibited by Azathioprine as an immunosuppressive agent and in the case of autoimmune patients (RA), TP3 caused a significant effect. Compared to TP5, TP3 was

more active on MN cells of tumor patients, while PMN cells reacted differently. MN and PMN cells isolated from head and neck tumor patients had a significantly higher adhesion capacity than those isolated from healthy individuals. TP3 significantly inhibited the adherence of MN and PMN cells, which is of therapeutic importance.

### Beneficial Effects

The Thymotridan therapy is based on the dose-effect relationship of the preparation used for different types of diseases with different doses.
1. To increase life expectancy, long-term (chronic) treatment is necessary, and then there is time
and, presumably, an opportunity the daily treatment that can be given at most (0.05 mg/kg body
weight/day) was designated as an effective dose (claims 2-6).
2. In cases where rapid, short-term (acute) powerful application is necessary and possible (in advanced stages of autoimmune and tumor diseases), then a maximal of 0.5 mg/kg body weight/day is optimal, and the required dose (Claims7).

In addition to the effect of the longer-term, physiological amount of treatment, the “enhancer” (hormone) effect also appeared at a dose several orders of lower level.

We claim:

1. Thymotridan, a pharmaceutical preparation containing a thymopentin analog (TP3/Arg-LysAsp)), is used to enhance the activity of the immune system in healthy persons or to causal
therapy of mammals with deficient immune activity to extend the lifespan of the mammal, to ensure increased immune activity in the preventive therapy of mammals, and to avoid infection
in young adults.

2. The use of the pharmaceutical preparation according to claim 1 in the preventive or causal therapy of a mammal with normal or deficient immune activity to extend the lifespan of the mammal, during the application to lengthen the immune activity by the “enhancer” compound,
Thymotridan, is given to a mammal, at a maximal daily dose 0.05 mg/kilogram/body weight.

3. The use of the pharmaceutical preparation according to claim 1 in the preventive therapy of an aging mammal with normal immune activity to extend the average age of the mammal, during which an “enhancer” compound that increases immune activity, Thymotridan, is given to a mammal in a maximal daily dose of 0.05 mg/kilogram/body weight, where it is a specific “enhancer” dose that increases and stimulates the number and activity of the cells involved in the defense mechanism of the mammal, preventing the unbalanced immune system.

4. The use of the pharmaceutical product according to claim 1 in the preventive therapy of a mammal with normal immune activity to ensure increased immune activity and to avoid infection in young people, during which application an immune activity-enhancing compound,
Thymotridan, is administered 0.05 mg/kg/body weight/daily with a specific “enhancer” dose that increases and stimulates the number and activity of cells involved in the defense mechanism
in the mammalian immune system.

5. The use of the pharmaceutical product according to claim 1 in the preventive therapy of a mammal with normal immune activity to ensure enhanced immune activity, to strengthen the defenses of patients who are about to undergo operations under anesthesia, during which application of an immune activity-enhancing compound, Thymotridan, is administered to a mammal at a “enhancer” dose 0.05 mg/kg/body weight/daily, which increases and stimulates
the number and activity of the cells in the immune system of the mammal involved in the defence mechanism to avoid post-traumatic diseases.

6. The use of the pharmaceutical product according to claim 1 in the therapy of a mammal with
unbalanced immune activity and through the renewal of the innate immune system (Monocyte,
macrophage), the number and activity of the missing immune cells are increased by application
Thymotridan “enhancer” compound increases the immune activity with 0.05 mg/kg/body weight/daily dose

7. The use of the pharmaceutical product according to claim 1 in the therapy of mammals, where the helper/suppressor cell ratio downregulated 1.0> the number of missing (mainly suppressor cells) is replaced through the renewal of the immune system (generally in autoimmune and tumour patients), where during the application Thymotridan “enhancer” compound increases the immune activity with 0.5 mg/kg/body weight/daily dose
